(19) United States
(12) Patent Application Publication
(10) Pub. No.: US 2022/0008533 A1
(43) Pub. Date: Jan. 13, 2022

(54) COMPOSITION AND METHODS OF TREATING INFLAMMATORY AND AUTOIMMUNE DISEASES

(71) Applicant: Tiziana Life Sciences PLC, London (GB)

(72) Inventor: Kunwar SHAILUBHAI, Line Lexington, PA (US)

(21) Appl. No.: 17/290,527

(22) PCT Filed: Oct. 31, 2019
(86) PCT No.: PCT/EP2019/079836

§ 371 Date: Apr. 30, 2021

Related U.S. Application Data
(60) Provisional application No. 62/753,445, filed on Oct. 31, 2018.

(51) Int. Cl. A61K 39/395; A61K 9/00; A61K 31/727; A61K 31/352; A61K 31/198; A61K 38/26; A61K 38/16; A61K 35/741; A61K 31/19; A61K 31/203; A61K 38/10

(52) U.S. Cl. CPC A61K 39/3955; A61K 9/0053; A61K 31/727; A61K 31/352; A61K 31/198; A61K 2039/542; A61K 38/164; A61K 35/741; A61K 31/19; A61K 31/203; A61K 38/10; A61K 38/26

(57) ABSTRACT

This invention relates to methods of treating disorders such as celiac disease by administrating anti-CD3 antibodies alone or in combination with additional agents.

### CROSS-REFERENCE TO RELATED APPLICATIONS

This application claims the benefit of priority to U.S. Provisional Application No. 62/753,445, filed Oct. 31, 2018, which is incorporated by reference herein in its entirety.

### FIELD OF THE INVENTION

This invention relates to oral formulations, dosages, and dosing regimens of anti-CD3 antibodies alone or in combination with compounds that improve gut barrier function and/or anti-inflammatory compound to treat inflammatory and autoimmune diseases including celiac disease (CeD) and Crohn's Disease (CD).

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: TIZI_018_001WO_SeqList_ST25.txt, date recorded: Oct. 29, 2019, file size ˜17,236 bytes).

### BACKGROUND OF THE INVENTION

Celiac disease (CeD) is an autoimmune disease which is related to dietary ingestion of gluten. Patients with celiac disease are severely sensitive to dietary ingestion of glutens that cause symptoms involving abdominal pain, bloating, diarrhea etc. Although etiology of celiac disease is only partially known, it is well-established that ingestion of gluten proteins present in wheat and other cereals are potentiators for the disease in individuals genetically predisposed to carrying the DQ2 or DQ8 human leukocyte antigen haplotypes. Proteomics and molecular studies has also shed some lights in the pathogenic role of tissue transglutaminase 2 (TG2) in CeD. This enzyme catalyzes conversion of gluten peptides to their deamidated metabolites which are highly potent to activate pathogenic T cells. In CeD patients TG2 is overexpressed. In addition, CeD patients, the intestinal barrier function is also disrupted due to alteration in microbiota and other inflammatory stimuli. The underlying mucosal inflammation also interferes with treatments to improve intestinal barrier function. One of the primary reasons for mucosal inflammation is dysregulation in functions of T regulatory cells (Tregs). Thus, a treatment comprising of combination of a compound that normalize or stimulate Tregs functions with a compound that improves intestinal barrier function may be appropriate for gastrointestinal (GI) inflammatory diseases such as CeD and CD.

DQ2 and DQ8 molecules serve as restriction elements for gluten-specific CD4+T lymphocytes, but are not sufficient to cause the chronic small intestinal inflammation. It is clear that ingestion of gluten protein is a prerequisite of disease development, resulting in villous flattening or crypt hyperplasia. Traditionally, gluten is a name of wheat proteins only, but gluten is now increasingly used as a term to denote proline- and glutamine (Gln)-rich proteins made by a class of grasses called triticeae, which mainly includes wheat, barley, and rye. A gluten-free diet reverses many disease manifestations, but it is not sufficient in a minority of patients with refractory CeD. Since patients having a refractory CeD and unusual clonal T-cell population (type II refractory CeD) are at risk for a particularly aggressive form of non-Hodgkin's lymphoma.

Thus a need exists for treatment modalities for celiac disease

### SUMMARY OF THE INVENTION

The invention provides methods for treating a disorder by administering an oral dosage form of an anti-CD3 antibody and a compound that improves gut barrier function and/or an anti-inflammatory compound. The compound that improves gut barrier function reduces gut permeability.

The disorder is an inflammatory disease, a gastrointestinal disorder, an autoimmune disease or cancer. The inflammatory disorder is an inflammatory disorder of the gastrointestinal tract. The gastrointestinal disorder is a gastrointestinal cancer. Alternatively, the gastrointestinal disorder is celiac disease, Crohn's disease, ulcerative colitis, irritable bowel syndrome, inflammatory bowel disease, or colon cancer. The autoimmune disease is for example, diabetes. The disorder is NASH, a bile acid disorder or a liver disease.

The compound that improves gut barrier function reduces gut permeability.

The a compound that improves gut barrier function is for example, a retinoic acid, a GC-C receptor agonist, heparin, lubiprostone, glutamine, GLP-2 peptide, a zonulin peptide inhibitor, a zonulin antagonist, larazotide, a probiotic bacteria or butyric acid.

The retinoic acid is all-trans-retinoic acid, 9-cis-retinoic acid, or 13-cis-retinoic acid. The retinoic acid is administered at a daily dose of about 15-45 mg/m². Preferably, the retinoic acid is administered at a daily dose of about 45 mg/m². Optionally, the daily dose is administered in two equally divided doses. For example, the retinoic acid is administered at a dose of about 22.5 mg/m² twice daily.

The GC-C receptor agonist is guanylin, uroguanylin, lymphoguanylin, heat-stable enterotoxin of E. coli (ST), linaclotide (e.g., Linzess®), plecanatide (e.g., Trulance®) or dolcanatide. The GC-C receptor agonist is administered at a daily dose of about 1 to about 10 mg. Preferably, the GC-C receptor agonist is administered at a daily dose of 3 mg or 6 mg. The GC-C agonist is administered once daily.

The anti-inflammatory compound is a monoclonal antibody specific for IL-17, TNF-α, α4-integrin, α4-β7 integrin, IL-12, or IL-23. Alternatively, the anti-inflammatory compound is Remicade®, a JNK inhibitor, a PI3K pathway inhibitor, an AKT inhibitor, a mTOR inhibitor, mesalamine, Uceris or a sphinosine-1-phosphate kinase inhibitor.

The anti-CD3 antibody is fully human, humanized or murine. The anti-CD3 antibody administered at a daily dose of about 0.1 mg to about 10 mg. The anti-CD3 antibody is administered for a treatment cycle of 30 days. For example, the anti-CD3 antibody is administered for a treatment cycle comprising an on period of 15 days and an off period of 15 days. In some aspects the anti-CD3 antibody and the compound that increases gut barrier function are administered for a treatment cycle of 30 days. In another aspect the anti-CD3 antibody and the compound that increases gut barrier function are administered for a treatment cycle, the treatment cycle comprising an on period of 15 days and an off period of 15 days for the anti-CD3 antibody and an on period of 30 days for the compound that increases gut barrier function. In a further aspect the anti-CD3 antibody, the compound that increases gut barrier function and the GC-C receptor agonist, are administered for a treatment cycle or 30 days. In another aspect the anti-CD3 antibody, the compound that increases gut barrier function and the GC-C receptor agonist, are administered for a treatment cycle, the treatment cycle comprising an on period of 15 days and an off period of 15 days for the anti-CD3 antibody or antigen-binding fragment thereof, an on period of 30 days for the GC-C agonist, and an on period of 30 days for the retinoic acid.

Optionally, each treatment cycle is repeated 2, 3, 4, 5, 6, 7, 8, 9, or 10 times.

The oral dosage form of the anti-CD3 antibody is an enteric polymer-coated oral dosage form such as a liquid-filled capsule or a powder-filed capsule. The enteric polymer-coated, liquid-filled oral capsule contains a liquid formulation having a unit dose of about 0.1 mg to 10 mg of an anti-CD3 antibody about 10 mM to about 500 mM sodium acetate trihydrate, about 10 mM to 500 mM sodium chloride, and about 0.01% to 1% polysorbate 80 (w/v). The enteric polymer-coated, powder-filled oral capsule contains a lyophilized powder formulation havinga unit dose of about 0.1 mg to 10 mg of an anti-CD3 antibody and one or more pharmaceutically acceptable excipients.

In some aspects the anti-CD3 antibody has a heavy chain comprising a variable heavy chain complementarity determining region 1 (VH CDR1) comprising the amino acid sequence of GYGMH (SEQ ID NO: 1), a variable heavy chain complementarity determining region 2 (VH CDR2) comprising the amino acid sequence of VIWYDGSKKYYVDSVKG (SEQ ID NO: 2), a variable heavy chain complementarity determining region 3 (VH CDR3) comprising the amino acid sequence of QMGYWHFDL (SEQ ID NO: 3); and a light chain comprising a variable light chain complementarity determining region 1 (VL CDR1) comprising the amino acid sequence of RASQSVSSYLA (SEQ ID NO: 4), a variable light chain complementarity determining region 2 (VL CDR2) comprising the amino acid sequence of DASNRAT (SEQ ID NO: 5), a variable light chain complementarity determining region 3 (VL CDR3) comprising the amino acid sequence of QQRSNWPPLT (SEQ ID NO: 6);

In another aspect the anti-CD3 antibody has a heavy chain comprising a VH CDR1 comprising the amino acid sequence SYGMH (SEQ ID NO: 7); a VH CDR2 comprising the amino acid sequence of IIWYDGSKKNYADSVKG (SEQ ID NO: 8); a VH CDR3 comprising the amino acid sequence of GTGYNWFDP (SEQ ID NO: 9); and a light chain comprising a VL CDR1 comprising the amino acid sequence of RASQSVSSSYLA (SEQ ID NO: 10), RASQGISSALA (SEQ ID NO: 11) or WASQGISSYLA (SEQ ID NO: 12); a VL CDR2 comprising the amino acid sequence of GASSRAT (SEQ ID NO: 13), YASSLQS (SEQ ID NO: 14), or DASSLGS (SEQ ID NO: 15); and a VL CDR3 comprising the amino acid sequence of QQYGSSPIT (SEQ ID NO: 16) or QQYYSTLT (SEQ ID NO: 17); and

In further aspects the anti-CD3 antibody has a heavy chain comprising a VH CDR1 comprising the amino acid sequence SYGMH (SEQ ID NO: 7); a VH CDR2 comprising the amino acid sequence of AIWYNGRKQDYADSVKG (SEQ ID NO: 18); a VH CDR3 comprising the amino acid sequence of GTGYNWFDP (SEQ ID NO: 9); and a light chain comprising a VL CDR1 comprising the amino acid sequence of RASQSVSSYLA (SEQ ID NO: 4) or RASQGISSALA (SEQ ID NO: 11); a VL CDR2 comprising the amino acid sequence of DASNRAT (SEQ ID NO: 5) or DASSLES (SEQ ID NO: 19); and a VL CDR3 comprising the amino acid sequence of QQRSNWPWT (SEQ ID NO: 20) or QQFNSYPIT (SEQ ID NO: 21).

The anti-CD3 antibody comprises a heavy chain variable region (VH) having the amino acid sequence of SEQ ID NOs: 22 (28F11 VH), 24 (23F10 VH), 26 (27H5 VH), or 32 (15C3 VH), and a light chain variable region (VL) having the amino acid sequence of SEQ ID NOs: 23 (28F111 VL), 25 (23F10 VL), 27 (27H5 VL1), 28 (27H5 VL2), 29 (27H5 VL3), 30 (27H5 VL4), 31 (27H5 VL5), 33 (15C3 VL1), or 34 (15C3 VL2).

In another aspect, the anti-CD3 antibody comprises a heavy chain variable region (VH) having the amino acid sequence of SEQ ID NO: 22 (28F11 VH) and a light chain variable region (VL) having the amino acid sequence of SEQ ID NO: 23 (28F11 VL).

In a further aspect, the anti-CD3 antibody comprises a heavy chain variable region (VH) having the amino acid sequence of SEQ ID NO: 24 (23F10 VH) and a light chain variable region (VL) having the amino acid sequence of SEQ ID NO: 25 (23F10 VL).

In yet another aspect the anti-CD3 antibody comprises a heavy chain variable region (VH) having the amino acid sequence of SEQ ID NO: 26 (27H5 VH) and a light chain variable region (VL) having the amino acid sequence of SEQ ID NOs: 27 (27H5 VL1), 28 (27H5 VL2), 29 (27H5 VL3), 30 (27H5 VL4), 31 (27H5 VL5).

In another aspect, the anti-CD3 thereof comprises a heavy chain variable region (VH) having the amino acid sequence of SEQ ID NO: 32 (15C3 VH), and a light chain variable region (VL) having the amino acid sequence of SEQ ID NOs: 33 (15C3 VL1) or 34 (15C3 VL2).

The anti-CD3 antibody or antigen-binding fragment thereof comprises a framework 2 region (FWR2) comprising the amino acid sequence WVRQAPGKGLEWV (SEQ ID NO: 35).

In some aspects, the anti-CD3 antibody or antigen-binding fragment thereof comprises a framework 3 region (FRW3) comprising the amino acid sequence RFTISRDNSKNTLYLQMNSLRAEDTAVYYCA (SEQ ID NO:36).

Preferably, the anti-CD3 antibody is an IgG1 isotype.

In some aspects, the anti-CD3 antibody or antigen-binding fragment thereof includes a mutation in the heavy chain at an amino acid residue at position 234, 235, 265, or 297 or combinations thereof, and reduces the release of cytokines from a T cell, wherein the amino acid residue positions are numbered according to Kabat. The mutation results in an alanine or glutamic acid residue at said position.

In another aspect, the anti-CD3 antibody or antigen-binding fragment thereof is an IgG1 isotype and contains at least a first mutation at position 234 and a second mutation at position 235, wherein said first mutation results in an alanine residue at position 234 and said second mutation results in a glutamic acid residue at position 235, wherein the amino acid residue positions are numbered according to Kabat.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 is an illustration showing the 3 phases of celiac disease.

FIG. 2 shows the dosage regimen for treatment with an anti CD3 antibody alone or in combination with other agents.

### DETAILED DESCRIPTION

The present invention provides method of treating a disorder by orally administering an antibody specific against CD3 epsilon chain (CD3F) alone or in combination a compound that improved gut barrier function and/or an anti-inflammatory compound. Disorders to be treated include inflammatory diseases (e.g., example inflammatory disorders of the GI tract), GI disorders, autoimmune disease (e.g. diabetes), cancer (e.g., GI cancer), NASH, bile disorders, or liver diseases. GI disorders includes celiac disease, Crohn's disease, ulcerative colitis, irritable bowel syndrome, inflammatory bowel disease, or colon cancer.

Specifically, the invention provides a method of treating CeD or CD by orally administering an antibody specific against CD3E alone or in combination as described herein.

Celiac Disease

It is widely accepted that fragments of gliadin, one of the main component of gluten, present in food pass through the epithelial barrier of the small intestine and enter the lamina propria where they are deaminated by tissue TG2 and then linked to specific HLA-class II (DQ2 or DQ8) molecules. The presence of T-cell receptors (TCRs) specific for the recognition of HLA-gliadin complexes lead to the IFNγ and interleukin 21 production, which favors the CD8+ T-cell cytotoxic activity accountable for the intestinal mucosal damage.

More recently, there has been directed attention toward alterations characterizing the T-helper 1 (TH1) CD4+ T-cell population in CeD. Tanscriptome next-generation RNA-sequencing (RNA-seq) analysis confirmed in CeD the potential importance of the proinflammatory cytokine IFN7, IL-17 and IL-15. Classically, IFN-7 is known to promote the CD4+ T-cells differentiation toward a profile of TH1-cytokines type, while it is known to inhibit the TH2 immune response and the regulatory T-cell survival. TH1-cells are known to develop cell-mediated immunity by enhancing and maintaining response of CD8+(cytotoxic) T cells and to activate the phagocyte-dependent inflammatory status. Moreover, CD4+ T cells have direct cytotoxic functions by secretion of cytotoxic granules containing granzyme B and perform and by a direct kill of target cells in an antigen (Ag)-HLA-class II fashion upon direct contact. Ag-presenting cells (i.e., dendritic cells or B-cells) present peptides derived from both exogenous Ags phagocytosed and processed in the endosomes and from endogenous processing via cell autophagy. TH1-cells also were known to promote B-cell Ig class switch and presentation of HLA.

Recent studies suggest a role for intraepithelial lymphocyte populations in the pathogenesis of celiac disease. An increase in intraepithelial lymphocytes has been known for many years as a characteristic feature of celiac disease, yet a role for these cells in disease pathogenesis has remained unclear. Intraepithelial lymphocytes appear to be the key cells in causing epithelial damage following activation. The activated lymphocytes may also change their characteristics from those typical of antigen-specific T cells to a phenotype of NK-like cells that are capable of mediating epithelial cell damage. It is also known that the expression of the cytokine interleukin (IL)-15 appears to be taking center stage in this process because IL-15 is up-regulated by epithelial cells and cells in the lamina propria in celiac disease and may be key factor contributing to altered properties of the intraepithelial lymphocyte population. The cytokine IL-15 is also known to interfere with anti-inflammatory function of T regulatory cells (Tregs). The depletion of Tregs and disruption of their function are probably the most important alterations in CeD patients. Therefore, upregulation of Tregs and improvement in intestinal barrier functions could be important for development of therapeutics for CeD.

A fundamental function of the intestinal epithelium is to act as a barrier that regulates interactions between the luminal contents, such as the intestinal microbiota, the underlying immune system, and the remainder of the body, while supporting vectorial transport of nutrients, water, and waste products. Intestinal barrier function requires a contiguous layer of epithelial cells as well as the junctions that seal the paracellular space between epithelial cells. In CeD patients, the barrier function is disrupted, gut permeability is enhanced and expression of tight junction proteins (occludins and ZO-1) is reduced, resulting in leaky gut. Compromised intestinal barrier function has been associated with a number of disease states, both intestinal and systemic. Although, the vast majority of these associations are merely correlative, but experimental evidence relating barrier dysfunction to disease pathogenesis exists in some cases, including inflammatory bowel disease (IBD), Crohn's Disease (CD) and celiac disease (CeD). Diminished intestinal barrier function has also been proposed to play a pathogenic role in CeD, as immune system exposure to gluten peptides is necessary for CeD to become clinically evident. However, the route by which gluten is passed from the lumen to the lamina propria is controversial and may involve either the transcellular or paracellular route. Thus, gut permeability and intestinal barrier integrity must be maintained to prevent CeD.

CDl03+ dendritic cells (DCs) act as migratory antigen-presenting cells and upon activation traffic to secondary lymphoid tissues, including the mesenteric lymph nodes (MLN) and Peyer's patches, carrying the antigenic material and live bacteria for presentation to adaptive immune cells. These migratory DCs promote immune tolerance through the differentiation of fork head box P3 (FOXP3⁺) Tregs cells. The production of this subset of Tregs is promoted by vitamin A analog retinoic acid in presence of TGF-beta. Furthermore, the production of retinoic acid by Intestinal epithelial cells (IEC)-conditioned CD-103⁺ DCs is responsible for the imprinting of gut-homing properties on T cells, allowing for the targeting of recirculating mature cells to the site of antigen encounter in the intestinal lamina propria. Thus, in addition to promoting naive T cell maturation based on antigen specificity, CD-103⁺ DCs relay the original context of antigenic encounter at the intestinal epithelial barrier. This process for production of Tregs is known to be stimulated by oral administration of anti-CD3 antibodies. Thus, oral administration with human anti-CD3 (Foralumab) is expected to promote production of Tregs that may produce anti-inflammatory activity to reduce gut inflammation in CeD patients.

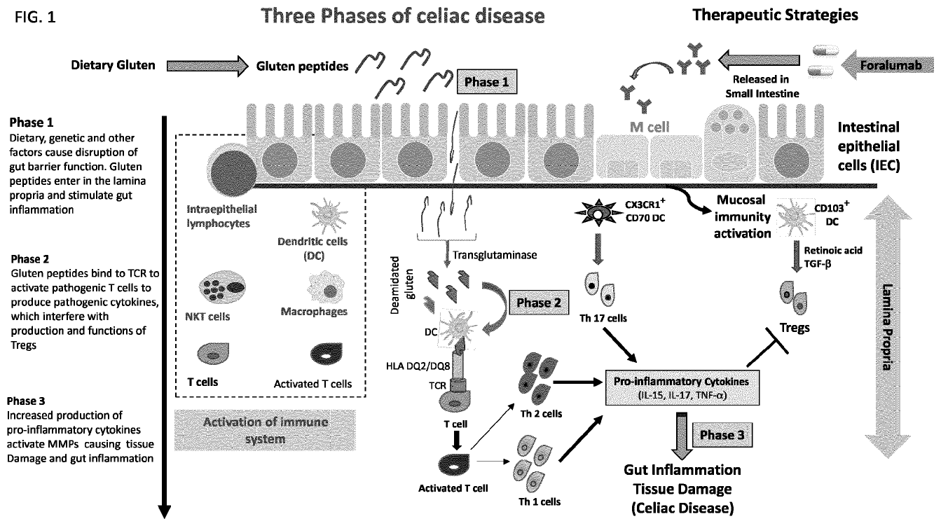

Therefore, the therapeutic strategy for CeD patients is shown in FIG. 1, encompass addressing three stages of CeD pathogenesis. The ingestion of gluten, followed by transport of gluten peptides in the lamina propria and their subsequent deamidation (Phase 1) may be reduced by improving barrier function. However, CeD patients already have gut inflammation with excessive production of pro-inflammatory cytokines (Phase 2) that may interfere with improvement of barrier function. Thus, improvement of barrier function is expected to be facilitated by increased production of Tregs. The production of Tregs can be enhanced by oral treatment with anti-CD3 antibody either alone or in combination with agents that improve gut barrier function and/or anti-inflammatory compounds. In addition, it is also anticipated that therapeutic modalities that improve gut barrier function might be synergistic with actions of anti-CD3 antibody. Thus, combination anti-CD3 with agents that improve gut barrier function and/or anti-inflammatory compounds may delay further progression of CeD. In addition, the anti-inflammatory actions of anti-CD3 will also reduce gut inflammation, which is not expected to reduce production of pro-inflammatory cytokines but also enhance functions of Tregs.

Also provided herein are methods of treating Crohn's disease, ulcerative colitis, irritable bowel syndrome, inflammatory bowel disease, or colon cancer by orally administering an antibody specific against CD3 epsilon chain (CD3ε) alone or in combination with a compound that improved gut barrier function and/or an anti-inflammatory compound. A recent study demonstrated that orally administered anti-CD3 antibody produces immunologic changes in ulcerative colitis patients (Boden et al., “Immunologic Alterations Associated with Oral Delivery of Anti-CD3 (OKT3) Monoclonal Antibodies in Patients with Moderate-to-Severe Ulcerative Colitis,” Crohn's & Colitis 360, vol. 1(2), July 2019, incorporated herein by reference in its entirety).

CD3 Antibodies

Antibodies specific for CD3 epsilon chain (CD3ε) and antigen binding fragments thereof are referred to herein as an anti-CD3 antibody. Any anti-CD3 antibody known in the art is suitable for use in the present invention. Anti-CD3 antibodies suitable for use in the present invention includes those disclosed in WO2005/118635, WO 2005/099755, and WO 2018/044948, the content of each are incorporated by reference in their entireties.

An exemplary anti-CD3 antibodies, comprise a heavy chain complementarity determining region 1 (CDRH1) comprising the amino acid sequence GYGMH (SEQ ID NO: 1), a heavy chain complementarity determining region 2 (CDRH2) comprising the amino acid sequence VIWYDGSKKYYVDSVKG (SEQ ID NO: 3), a heavy chain complementarity determining region 3 (CDRH3) comprising the amino acid sequence QMGYWHFDL (SEQ ID NO: 4), a light chain complementarity determining region 1 (CDRL1) comprising the amino acid sequence RASQSVSSYLA (SEQ ID NO: 5), a light chain complementarity determining region 2 (CDRL2) comprising the amino acid sequence DASNRAT (SEQ ID NO: 6), and a light chain complementarity determining region 3 (CDRL3) comprising the amino acid sequence QQRSNWPPLT (SEQ ID NO: 7).

In some embodiments, the anti-CD3 antibody comprises a variable heavy chain amino acid sequence comprising

(SEQ ID NO: 8)
QVQLVESGGGVVQPGRSLRLSCAASGFKFSGYGMHWVRQAPGKGLEWVA
VIWYDGSKKYYVDSVKGRFTISRDNSKNTLYLQMNSLRAEDTAVYYCAR
QMGYWHFDLWGRGTLVTVSS

and a variable light chain amino acid sequence comprising

(SEQ ID NO: 9)
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPLTFG
GGTKVEIK.

Preferably, the anti-CD3 antibody comprises a heavy chain amino acid sequence comprising:

(SEQ ID NO: 10)
QVQLVESGGGVVQPGRSLRLSCAASGFKFSGYGMHWVRQAPGKGLEWVAV
IWYDGSKKYYVDSVKGRFTISRDNSKNTLYLQMNSLRAEDTAVYYCARQM
GYWHFDLWGRGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPEAEGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK

and a light chain amino acid sequence
comprising:

(SEQ ID NO: 11)
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPLTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

This anti-CD3 antibody is referred to herein as NI-0401, Foralumab, or 28F11-AE. (see e.g., Dean Y, Dépis F, Kosco-Vilbois M. “Combination therapies in the context of anti-CD3 antibodies for the treatment of autoimmune diseases.” Swiss Med Wkly. (2012) (the contents of which are hereby incorporated by reference in its entirety).

In some embodiments, the anti-CD3 antibody includes a full length anti-CD3 antibody. In alternative embodiments, the anti-CD3 antibody includes an antibody fragment that specifically binds CD3. In some embodiments, the anti-CD3 antibody formulation includes a combination of full-length anti-CD3 antibodies and antigen binding fragments that specifically bind CD3.

In some embodiments, the antibody or antigen-binding fragment thereof that binds CD3 is a monoclonal antibody, domain antibody, single chain, Fab fragment, a F(ab′)₂ fragment, a scFv, a scAb, a dAb, a single domain heavy chain antibody, or a single domain light chain antibody. In some embodiments, such an antibody or antigen-binding fragment thereof that binds CD3 is a mouse, other rodent, chimeric, humanized or fully human monoclonal antibody.

Optionally, the anti-CD3 antibody or antigen binding fragment thereof used in the formulations of the disclosure includes at least one an amino acid mutation. Typically, the mutation is in the constant region. The mutation results in an antibody that has an altered effector function. An effector function of an antibody is altered by altering, i.e., enhancing or reducing, the affinity of the antibody for an effector molecule such as an Fc receptor or a complement component. For example, the mutation results in an antibody that is capable of reducing cytokine release from a T-cell. For example, the mutation is in the heavy chain at amino acid residue 234, 235, 265, or 297 or combinations thereof. Preferably, the mutation results in an alanine residue at either position 234, 235, 265 or 297, or a glutamate residue at position 235, or a combination thereof.

Preferably, the anti-CD3 antibody provided herein contains one or more mutations that prevent heavy chain constant region-mediated release of one or more cytokine(s) in vivo.

In some embodiments, the anti-CD3 antibody or antigen binding fragment thereof used in the formulations of the disclosure is a fully human antibody. The fully human CD3 antibodies used herein include, for example, a L²³⁴ L²³⁵→A²³⁴ E²³⁵ mutation in the Fc region, such that cytokine release upon exposure to the anti-CD3 antibody is significantly reduced or eliminated. The L²³⁴ L²³⁵→A²³⁴ E²³⁵ mutation in the Fc region of the anti-CD3 antibodies provided herein reduces or eliminates cytokine release when the anti-CD3 antibodies are exposed to human leukocytes, whereas the mutations described below maintain significant cytokine release capacity. For example, a significant reduction in cytokine release is defined by comparing the release of cytokines upon exposure to the anti-CD3 antibody having a L²³⁴ L²³⁵→A²³⁴ E²³⁵ mutation in the Fc region to level of cytokine release upon exposure to another anti-CD3 antibody having one or more of the mutations described below. Other mutations in the Fe region include, for example, L²³⁴ L²³⁵→A²³⁴ A²³⁵, L²³⁵→E²³⁵, N²⁹⁷→A²⁹⁷, and D²⁶⁵→A²⁶⁵.

The term “cytokine” refers to all human cytokines known within the art that bind extracellular receptors expressed on the cell surface and thereby modulate cell function, including but not limited to IL-2, IFN-gamma, TNF-a, IL-4, IL-5, IL-6, IL-9, IL-10, and IL-13.

Agents that Improve Gut Barrier Function

Agents that improve gut barrier function include any agent that improves at least on gut barrier function such as a reduction of gut permeability.

Exemplary agents that impoved gut barrier function include for example, retinoic acid, a GC-C receptor agonist, heparin, lubiprostone, glutamine, GLP-2 peptide, a zolulin peptide inhibitor, a zonulin antagonist, larazotide, a probiotic bacteria or butyric acid.

Retinoic acids includes such as all-trans-retinoic acid, 9-cis-retinoic acid, or 13-cis-retinoic acid.

A GC-C receptor agonist includes for example, guanylin, uroguanylin, lymphoguanylin, heat-stable enterotoxin of E. coli (ST), linaclotide, plecanatide or dolcanatide. Preferably, the GC-C receptor agonist is Linzess® or Trulance®.

Additional, agents that improve gut barrier function and or reduce gut permeability include for example, non-digestible carbohydrates, such as fructo-oligosaccharides (FOS), galacto-oligosaccharides (GOS), inulin, Arabic gum, xylo-oligosaccharides, resistant starch and the like and LC-PUFA, such as arachidonic acid (AA) or docosahexanoic acid (DHA) and optionally Human milk oligosaccharides, such as sialyllactose and/or Gangliosides such as those contained in delactosed whey from buffalo milk, and/or Milk or colostrum fractions, such as acid, rennet or micellar casein, acid, sweet or ultra whey, fat globules membranes and the like, and/or Extensive hydrolysed protein, such as those obtained from whey protein hydrolysis, and/or Polyamines such as spermine or spermidine and/or one or more polyamine precursors, in particular ornithine and arginine

Preferably, non-digestible carbohydrates may be selected in the group of fructo-oligosaccharides, galacto-oligosaccharides, sialo-oligosaccharides, xylo-oligosaccharides, inulin, arabic gum, guar gum, resistant starch and/or milk-derived oligosaccharides and be added to the microorganism cocktail. One or more of these can be used in the total doses of from about 0.01 to 5 g/100 ml, and preferably 1-2 g/100 ml. A mixture of two or more carbohydrates may be used, each carbohydrate ranging between the 5% to 95% of the carbohydrate mixture.

Preferably, particular lipids may be used. For instance, an effective amount of at least one n-6 polyunsaturated fatty acid in combination with at least one n-3 polyunsaturated fatty acid, such as C20 or C22 n-6 fatty acid and one C20 or C22 n-3 fatty acid. The C20 or C22, n-6 fatty acid is present in a total amount of about 0.01 to 6.0% by weight of all fatty acids in the composition, preferably in a total amount of 0.1 to 1%. The C20 or C22 n-3 fatty acid is included in a total amount of about 0.01 to about 6.0% by weight of all fatty acids in the composition, preferably in a total amount of 0.1 to 1%. Preferably, the n-6 polyunsaturated fatty acid used in the present invention is arachidonic acid (AA, C20:4 n-6) and the n-3 polyunsaturated fatty acid used in the present invention is docosahexanoic acid (DHA, C22:6, n-3). The effective AA:DHA ratio is about 1:1 to 2.5:1, and preferably 1:1 to 2:1. The source of the LC-PUFA may be egg lipids, fungal oil, low EPA fish oil, algal oil, etc.

Gangliosides, a second class of lipids, may also be added to the combination of ingredients, for example in an amount of from about 1-20 microMol/L, and preferably 6-15 microMol/L. The source of gangliosides may be cow's milk, cow's colostrum, but preferably buffalo's milk, milk serum or colostrum, goat's milk, colostrum or serum and/or derivatives of either.

The combination may also contain polyamines, in particular spermidine, spermine, or putrescine and/or one or more polyamine precursors, in particular ornithine and arginine. They can be used in an amount of about 10 to 2,000 microg/100 g solid formula. The polyamine is preferably at least two or more selected from the group consisting of spermine, spermidine, putrescine and cadaverine. Preferably the composition comprises about 10-90% of spermine, 10-90% of spermidine, 0-90% of putrescine and 0-20% of cadaverine.

Preferably, the milk fractions (enriched in growth factors) may be in the form of fat globule membrane proteins, acid, rennet or micellar casein, acid, sweet or ultra whey, whey protein hydrolysates, for example. They can be used in an amount of about 0.01 to 7 g /100 ml, and preferably 0.5-3 g/100 ml.

Anti-Inflammatory Compounds

Anti-inflammatory compound is any substance that reduces inflammation or swelling. An anti-inflammatory compound is for example a monoclonal antibody, such as a monoclonal antibody specific for IL-17, TNF-a, α4-integrin, α4-β7 integrin, IL-12, or IL-23. Preferred, monoclonal antibodies include Humira®, Tysabri®, Entyvio®, or Stelara®. Additional ant inflammatory compound include Remicade®, a JNK inhibitor, a PI3K pathway inhibitor, an AKT inhibitor, a mTOR inhibitor, mesalamine, budesonide (Uceris®) or a sphinosine-1-phosphate kinase inhibitor.

Antibody Formulations

The antibody formulations comprises a unit dose of the antibody in the range of: about 0.1 mg to about 50 mg; about 0.1 mg to about 25 mg; or 0.1 mg to about 10 mg. For example, the unit dose is about 0.1, 0.2, 0.3, 0.4, 0.5, 0.6, 0.7, 0.8, 0.9, 1.0, 1.5, 2.0, 2.5, 3.0, 3.5, 4.0, 5.0, 5.5, 6.0, 6.5, 7.0, 7.5, 8.0, 8.5, 9, 9.5, 10 mg or more. Preferably, the unit dose is 0.5 mg, 2.5 mg or 5.0 mg. Preferably, the antibody is formulated for oral administration. Preferred oral antibody formulation include Foralumab, Adalimumab, Infliximab, Nataslizumab, Vedolizumab, and Ustekinumab.

The antibody formulation can be a liquid. For example the liquid formulation is aqueous. Alternatively, the antibody formulation is a lyophilized powder. When the antibody formulation is a lyophilized powder, additionally bulking agent may be added to provide adequate structure to the lyophilized cake. This additional bulking agent may increase the stability of the lyophilized cake upon storage. Alternatively, this additional bulking agent may aide in the production of the dosage form, e.g., oral capsule. Bulking against are described herein and include polyols such as, trehalose, mannitol, maltose, lactose, sucrose, sorbitol, or glycerol, starch, microcrystalline cellulose, low moisture microcrystalline cellulose such as Avicel or polethylen glycols (PEG). The antibody formulation is suitable for oral administration. The oral capsule may be enteric coated to minimize exposure to stomach acidity.

The anti-CD3 antibody formulation includes one or more salts (a buffering salt), one or more polyols and one or more excipients. The formulations of the present invention may also contain buffering agents, or preservatives. The anti-CD3 antibody formulation is buffered in a solution at a pH in the range of about 4 to 8; in the range of about 4 to 7; in the range of about 4 to 6; in the range of about 5 to 6; or in the range of about 5.5 to 6.5. Preferably, the pH is 5.5.

Examples of salts include those prepared from the following acids: hydrochloric, hydrobromic, sulfuric, nitric, phosphoric, maleic, acetic, salicylic, citric, boric, formic, malonic, succinic, and the like. Such salts can also be prepared as alkaline metal or alkaline earth salts, such as sodium, potassium or calcium salts. Examples of buffering agents include phosphate, citrate, acetate, and 2-(N-morpholino) ethanesulfonic acid (MES).

The formulations of the present invention may include a buffer system. As used in this application, the terms “buffer” or “buffer system” is meant a compound that, usually in combination with at least one other compound, provides a buffering system in solution that exhibits buffering capacity, that is, the capacity to neutralize, within limits, either acids or bases (alkali) with relatively little or no change in the original pH.

Buffers include borate buffers, phosphate buffers, calcium buffers, and combinations and mixtures thereof. Borate buffers include, for example, boric acid and its salts, for example, sodium borate or potassium borate. Borate buffers also include compounds such as potassium tetraborate or potassium metaborate that produce borate acid or its salt in solutions.

A phosphate buffer system includes one or more monobasic phosphates, dibasic phosphates and the like. Particularly useful phosphate buffers are those selected from phosphate salts of alkali and/or alkaline earth metals. Examples of suitable phosphate buffers include one or more of sodium dibasic phosphate (Na2HPO4), sodium monobasic phosphate (NaH2PO4) and potassium monobasic phosphate (KH2PO4). The phosphate buffer components frequently are used in amounts from 0.01% or to 0.5% (w/v), calculated as phosphate ion.

Other known buffer compounds can optionally be added to the according to the CD3 formulations, for example, citrates, sodium bicarbonate, TRIS, and the like. Other ingredients in the solution, while having other functions, may also affect the buffer capacity. For example, EDTA, often used as a complexing agent, can have a noticeable effect on the buffer capacity of a solution.

Preferred salts for use in the formulation of the invention include sodium chloride, sodium acetate, sodium acetate trihydrate and sodium citrate.

The concentration of salt in the formulations according to the invention is between about 10 mM and 500 mM, between about 25 m and 250 mM, between about 25 nM and 150 mM.

The sodium acetate trihydrate is at a concentration in the range of about 10 mM to 100 mM. For example, the sodium acetate trihydrate is at about 10, 15, 20, 25, 30, 35, 40, 45, 50, 55, 60, 65, 70, 75, 80, 85, 90, 95 or 100 mM. Preferably, the sodium acetate trihydrate is at 25 mM.

The sodium chloride at a concentration in the range of about 50 mM to 500 mM. For example, the sodium chloride is at about 50, 55, 60, 65, 70, 75, 80, 85, 90, 95, 100, 125, 150, 175, 200, 225, 250, 275, 300, 325, 350, 375, 400, 425, 450, 475 or 500 mM. Preferably, the sodium chloride is at a concentration of about 125 mM.

The sodium citrate is at a concentration in the range of about 10 mM to 100 mM For example the sodium citrate is at about 10, 15, 20, 25, 30, 35, 40, 45, 50, 55, 60, 65, 70, 75, 80, 85, 90, 95 or 100 mM. Preferably, the sodium citrate is in the range of about 25 to 50 mM.

In some embodiments, the salt is sodium acetate trihydrate at a concentration in the range of about 25 mm to 100 mm and sodium chloride at a concentration in the range of about 150 mm to 500 mm.

Preferably, the formulation includes about 25 mM sodium acetate trihydrate and about 150 mM sodium chloride.

The formulation includes one or more polyols as a bulking agent and/or stabilizing excipients. Polyols include for example, trehalose, mannitol, maltose, lactose, sucrose, sorbitol, or glycerol. The polyols is at a concentration in the range of about 0.1% to 50% or 5% to 25%. For example, the polyol is at about 1, 2, 3, 4, 5, 10, 15, 20, 25, 30, 35, 40, 45 or 50%

In some embodiments, the polyol is trehalose at a concentration in the range of about 1% to 50% or 5% to 25%. For example, the trehalose is at about 1, 2, 3, 4, 5, 10, 15, 20, 25, 30, 35, 40, 45 or 50%. Preferably the trehalose is at a concentration of about 10% or about 20%. Most preferably, the trehalose is at a concentration of about 20%.

In some embodiments, the polyol is sorbitol at a concentration in the range of about 1% to about 10%. In some embodiments, the polyol is glycerol at a concentration in the range of about 1% to about 10%.

In some embodiments, the polyol is mannitol at a concentration in the range of about 0.1% to about 10%. In some embodiments, the polyol is maltose at a concentration in the range of about 1% to about 10%.

The formulation includes one or more excipients and/or surfactants to suppress or otherwise reduce antibody aggregation. Suitable excipients to reduce antibody aggregation include, by way of non-limiting example, a surfactant such as, by way of non-limiting example, Polysorbate 20 or Polysorbate 80. In some embodiments, the Polysorbate 20 or Polysorbate 80 is present at a concentration in the range of about 0.01 to 1% or about 0.01 to 0.05%. For example the Polysorbate 20 or Polysorbate 80 is at a concentration of about 0.01. 0.02, 0.03, 0.04, 0.05, 0.06, 0.07. 0.08, 0.09, 0.1, 0.2, 0.3. 0.4, 0.5, 0.6, 0.7, 0.8. 0.9, or 1.0%.

Preferably the surfactant is Polysorbate 80 at a concentration in the range of about 0.01 to 0.05%. More preferably, the Polysorbate 80 is at 0.02%.

The formulation includes one or more excipients to reduce antibody oxidation. Suitable excipients to reduce antibody oxidation include, by way of non-limiting example, antioxidants. Antioxidants include for example, methionine, D-arginine, BHT or ascorbic acid. The antioxidant is present at a concentration in the range of about 0.01% to 1%; 0.1% to 1%; or 0.1% to 0.5%. In some embodiments, the antioxidant is methionine. In some embodiments, the methionine is present at a concentration in the range of about 0.01% to 1%; 0.1% to 1%; or 0.1% to 0.5%. For example, the methionine is present at a concentration of about 0.01. 0.02, 0.03, 0.04, 0.05, 0.06, 0.07. 0.08, 0.09, 0.1, 0.2, 0.3. 0.4, 0.5, 0.6, 0.7, 0.8. 0.9, or 1.0%. Preferably, the methionine is at about 0.1%.

The formulation includes one or more chelating agents, such as for example ethylenediaminetetraacetic acid (EDTA). The chelating agent is at a concentration in the range of 0.01% to 1%; 0.1% to 1%; or 0.1% to 0.5%. For example, the chelating agent is present at a concentration of about 0.01. 0.02, 0.03, 0.04, 0.05, 0.06, 0.07. 0.08, 0.09, 0.1, 0.2, 0.3. 0.4, 0.5, 0.6, 0.7, 0.8. 0.9, or 1.0%. Preferably, the chelating agent is EDTA at a concentration of about 0.1%.

In some embodiments, the formulation includes one or more excipients to increase stability. In some embodiments, the excipient to increase stability is human serum albumin. In some embodiments, the human serum albumin is present in the range of about 1 mg to about 5 mg.

In some embodiments, the formulation includes magnesium stearate (Mg stearate), an amino acid, or both mg-stearate and an amino acid. Suitable amino acids include for example, leucine, arginine, histidine, or combinations thereof.

In some embodiments the one or more additional excipients is low moisture microcrystalline cellulose, such as Avicel, polyethylene glycols (PEG), or a starch.

Further examples of pharmaceutically acceptable carriers and excipients useful for the formulations of the present invention include, but are not limited to binders, fillers, disintegrants, lubricants, anti-microbial agents, antioxidant, and coating agents such as: BINDERS: corn starch, potato starch, other starches, gelatin, natural and synthetic gums such as acacia, xanthan, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone (e.g., povidone, crospovidone, copovidone, etc), methyl cellulose, Methocel, pre-gelatinized starch (e.g., STARCH 1500® and STARCH 1500 LM®, sold by Colorcon, Ltd.), hydroxypropyl methyl cellulose, microcrystalline cellulose (FMC Corporation, Marcus Hook, Pa., USA), Emdex, Plasdone, or mixtures thereof, FILLERS: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, dextrose, fructose, honey, lactose anhydrate, lactose monohydrate, lactose and aspartame, lactose and cellulose, lactose and microcrystalline cellulose, maltodextrin, maltose, mannitol, microcrystalline cellulose &amp; guar gum, molasses, sucrose, or mixtures thereof, DISINTEGRANTS: agar-agar, alginic acid, calcium carbonate, microcrystalline cellulose, croscarmellose sodium, crospovidone, polacrilin potassium, sodium starch glycolate, (such as Explotab), potato or tapioca starch, other starches, pre-gelatinized starch, clays, other algins, other celluloses, gums (like gellan), low-substituted hydroxypropyl cellulose, ployplasdone, or mixtures thereof, LUBRICANTS: calcium stearate, magnesium stearate, mineral oil, light mineral oil, glycerin, sorbitol, mannitol, polyethylene glycol, other glycols, compritol, stearic acid, sodium lauryl sulfate, sodium stearyl fumarate, (such as Pruv), vegetable based fatty acids lubricant, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Deaussa Co., Piano, Tex. USA), a pyrogenic silicon dioxide (CAB-O-SIL, Cabot Co., Boston, Mass. USA), or mixtures thereof, ANTI-CAKING AGENTS: calcium silicate, magnesium silicate, silicon dioxide, colloidal silicon dioxide, talc, or mixtures thereof, ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, or mixtures thereof, ANTOXIDANTS: ascorbic acid, BHA, BHT, EDTA, or mixture thereof, and COATING AGENTS: sodium carboxymethyl cellulose, cellulose acetate phthalate, ethylcellulose, gelatin, pharmaceutical glaze, hydroxypropyl cellulose, hydroxypropyl methylcellulose (hypromellose), hydroxypropyl methyl cellulose phthalate, methylcellulose, polyethylene glycol, polyvinyl acetate phthalate, shellac, sucrose, titanium dioxide, carnauba wax, microcrystalline wax, gellan gum, maltodextrin, methacrylates, microcrystalline cellulose and carrageenan or mixtures thereof.

The formulation can also include other excipients and categories thereof including but not limited to Pluronic®, Poloxamers (such as Lutrol® and Poloxamer 188), ascorbic acid, glutathione, protease inhibitors (e.g. soybean trypsin inhibitor, organic acids), pH lowering agents, creams and lotions (like maltodextrin and carrageenans); materials for chewable tablets (like dextrose, fructose, lactose monohydrate, lactose and aspartame, lactose and cellulose, maltodextrin, maltose, mannitol, microcrystalline cellulose and guar gum, sorbitol crystalline); parenterals (like mannitol and povidone); plasticizers (like dibutyl sebacate, plasticizers for coatings, polyvinylacetate phthalate); powder lubricants (like glyceryl behenate); soft gelatin capsules (like sorbitol special solution); spheres for coating (like sugar spheres); spheronization agents (like glyceryl behenate and microcrystalline cellulose); suspending/gelling agents (like carrageenan, gellan gum, mannitol, microcrystalline cellulose, povidone, sodium starch glycolate, xanthan gum); sweeteners (like aspartame, aspartame and lactose, dextrose, fructose, honey, maltodextrin, maltose, mannitol, molasses, sorbitol crystalline, sorbitol special solution, sucrose); wet granulation agents (like calcium carbonate, lactose anhydrous, lactose monohydrate, maltodextrin, mannitol, microcrystalline cellulose, povidone, starch), caramel, carboxymethylcellulose sodium, cherry cream flavor and cherry flavor, citric acid anhydrous, citric acid, confectioner's sugar, D&C Red No. 33, D&C Yellow #10 Aluminum Lake, disodium edetate, ethyl alcohol 15%, FD&C Yellow No. 6 aluminum lake, FD&C Blue #1 Aluminum Lake, FD&C Blue No. 1, FD&C blue no. 2 aluminum lake, FD&C Green No. 3, FD&C Red No. 40, FD&C Yellow No. 6 Aluminum Lake, FD&C Yellow No. 6, FD&C Yellow No.10, glycerol palmitostearate, glyceryl monostearate, indigo carmine, lecithin, mannitol, methyl and propyl parabens, mono ammonium glycyrrhizinate, natural and artificial orange flavor, pharmaceutical glaze, poloxamer 188, Polydextrose, polysorbate 20, polysorbate 80, polyvidone, pregelatinized corn starch, pregelatinized starch, red iron oxide, saccharin sodium, sodium carboxymethyl ether, sodium chloride, sodium citrate, sodium phosphate, strawberry flavor, synthetic black iron oxide, synthetic red iron oxide, titanium dioxide, and white wax.

In some embodiments the antibody formulation is a liquid and the concentration of sodium acetate is about 10 mM to 500 mM; the concentration of sodium chloride is about 10 mM to 500 mM; the concentration of polysorbate 80 is about 0.01% to 1% (w/v); the concentration of trehalose is about 5% to 50% (w/v); and the concentration of methionine is 0.01% to 1% (w/v). Optionally, the formulation further includes EDTA at the concentration of about 0.01% to 1% (w/v). The unit dose of the anti-CD3 antibody or antigen binding fragment thereof is in the range of about 0.1 mg to 10 mg. In some embodiments the liquid formulation is lyophilized to form a powder.

In some embodiments the antibody formulation is a liquid and contains 25 mM sodium acetate, 125 mM sodium chloride, 0.02% polysorbate 80 (w/v), 20% trehalose (w/v), 0.1% methionine (w/v) and a unit dose of the anti-CD3 antibody or antigen binding fragment thereof in the range of about 0.1 mg to 10 mg. Optionally, the formulation further includes 0.1% EDTA (w/v). In some embodiments the liquid formulation is lyophilized to form a powder.

In a specific embodiment, the liquid antibody formulation includes 25 mM sodium acetate, 125 mM sodium chloride, 0.02% polysorbate 80 (w/v), 20% trehalose (w/v), 0.1% methionine (w/v) and a 0.5 mg unit dose of the anti-CD3 antibody or antigen binding fragment. Also included in the invention is a lyophilized powder of this formulation.

In a specific embodiment, the liquid antibody formulation includes 25 mM sodium acetate, 125 mM sodium chloride, 0.02% polysorbate 80 (w/v), 20% trehalose (w/v), 0.1% methionine (w/v) and a 0.2.5 mg unit dose of the anti-CD3 antibody or antigen binding fragment. Also included in the invention is a lyophilized powder of this formulation.

In a specific embodiment, the liquid antibody formulation includes 25 mM sodium acetate, 125 mM sodium chloride, 0.02% polysorbate 80 (w/v), 20% trehalose (w/v), 0.1% methionine (w/v) and a 5.0 mg unit dose of the anti-CD3 antibody or antigen binding fragment. Also included in the invention is a lyophilized powder of this formulation.

In a specific embodiment, the liquid antibody formulation includes 25 mM sodium acetate, 125 mM sodium chloride, 0.02% polysorbate 80 (w/v), 20% trehalose (w/v), 0.1% methionine (w/v), 0.1% EDTA (w/v) and a 0.5 mg unit dose of the anti-CD3 antibody or antigen binding fragment. Also included in the invention is a lyophilized powder of this formulation.

In a specific embodiment, the liquid antibody formulation includes 25 mM sodium acetate, 125 mM sodium chloride, 0.02% polysorbate 80 (w/v), 20% trehalose (w/v), 0.1% methionine (w/v), 0.1% EDTA (w/v) and a 0.2.5 mg unit dose of the anti-CD3 antibody or antigen binding fragment. Also included in the invention is a lyophilized powder of this formulation.

In a specific embodiment, the liquid antibody formulation includes 25 mM sodium acetate, 125 mM sodium chloride, 0.02% polysorbate 80 (w/v), 20% trehalose (w/v), 0.1% methionine (w/v), 0.1% EDTA (w/v) and a 5.0 mg unit dose of the anti-CD3 antibody or antigen binding fragment. Also included in the invention is a lyophilized powder of this formulation.

In some embodiments the formulation is a lyophilized powder where the ratio of anti-CD3 antibody or antigen binding fragment to polysorbate 80 is about 1:0.01 to 0.1 (w/w); the ratio of anti-CD3 antibody or antigen binding fragment trehalose is about 1:10 to 50 (w/w); the ratio of anti-CD3 antibody or antigen binding fragment methionine about 1:0.1 to 0.5 (w/w); the ratio of anti-CD3 antibody or antigen binding fragment sodium acetate is about 1:0.1 to 1.0 (w/w); and the ratio of anti-CD3 antibody or antigen binding fragment sodium chloride is about 1:0.5 to 2.0 (w/w). Optionally, the formulation further includes EDTA where the ratio of anti-CD3 antibody or antigen binding fragment to: EDTA is about 1:0.1 to 0.5 (w/w). The unit dose of the anti-CD3 antibody or antigen binding fragment thereof is in the range of about 0.1 mg to 10 mg.

In some embodiments, the antibody formulation is a powder, e.g., a lyophilized powder having a unit dose of about 0.1 mg to 10 mg of an anti-CD3 antibody or antigen binding fragment thereof and about 0.58 mg of sodium acetate trihydrate, about 1.25 mg sodium chloride, about 0.034 mg polysorbate 80, about 34 mg trehalose and about 0.17 mg methionine per 1 mg of anti-CD3 antibody or antigen binding fragment thereof. Optionally, the powder formulation further included 0.17 mg EDTA per 1 mg of anti-CD3 antibody or antigen binding fragment thereof. Preferably, the unit dose is 0.5 mg, 2.5 mg or 5.0 mg.

In a specific embodiment, the antibody formulation is a powder, e.g., a lyophilized powder having a unit dose of about 0.5 mg of an anti-CD3 antibody or antigen binding fragment thereof and about 0.58 mg of sodium acetate trihydrate, about 1.25 mg sodium chloride, about 0.034 mg polysorbate 80, about 34 mg trehalose and about 0.17 mg methionine per 1 mg of anti-CD3 antibody or antigen binding fragment thereof.

In a specific embodiment, the antibody formulation is a powder, e.g., a lyophilized powder having a unit dose of about 2.5 mg of an anti-CD3 antibody or antigen binding fragment thereof and about 0.58 mg of sodium acetate trihydrate, about 1.25 mg sodium chloride, about 0.034 mg polysorbate 80, about 34 mg trehalose and about 0.17 mg methionine per 1 mg of anti-CD3 antibody or antigen binding fragment thereof.

In a specific embodiment, the antibody formulation is a powder, e.g., a lyophilized powder having a unit dose of about 5 mg of an anti-CD3 antibody or antigen binding fragment thereof and about 0.58 mg of sodium acetate trihydrate, about 1.25 mg sodium chloride, about 0.034 mg polysorbate 80, about 34 mg trehalose and about 0.17 mg methionine per 1 mg of anti-CD3 antibody or antigen binding fragment thereof.

In a specific embodiment, the antibody formulation is a powder, e.g., a lyophilized powder having a unit dose of about 0.5 mg of an anti-CD3 antibody or antigen binding fragment thereof and about 0.58 mg of sodium acetate trihydrate, about 1.25 mg sodium chloride, about 0.034 mg polysorbate 80, about 34 mg trehalose, about 0.17 mg EDTA and about 0.17 mg methionine per 1 mg of anti-CD3 antibody or antigen binding fragment thereof.

In a specific embodiment, the antibody formulation is a powder, e.g., a lyophilized powder having a unit dose of about 2.5 mg of an anti-CD3 antibody or antigen binding fragment thereof and about 0.58 mg of sodium acetate trihydrate, about 1.25 mg sodium chloride, about 0.034 mg polysorbate 80, about 34 mg trehalose, about 0.17 mg EDTA and about 0.17 mg methionine per 1 mg of anti-CD3 antibody or antigen binding fragment thereof.

In a specific embodiment, the antibody formulation is a powder, e.g., a lyophilized powder having a unit dose of about 5 mg of an anti-CD3 antibody or antigen binding fragment thereof and about 0.58 mg of sodium acetate trihydrate, about 1.25 mg sodium chloride, about 0.034 mg polysorbate 80, about 34 mg trehalose, about 0.17 mg EDTA and about 0.17 mg methionine per 1 mg of anti-CD3 antibody or antigen binding fragment thereof.

The moisture (i.e., water) content of the formulations according to the invention (either in a liquid, lyophilized or final dosage form (e.g. capsule) is less than about 7%, 6%, 5%, 4%, 3%, 2% or 1%. Preferably, the moisture content is in the range of 2-5%, more preferably the moisture content is in the range of 1-2%, most preferably, the moisture content is less than 1%. Methods of determining moisture content is known in the art, for example moisture content is determined by Karl Fischer titration.

In some embodiments, the osmolality of the formulation is about 800-950 (e.g., about 825-925) mOsm/kg.

The antibody formulations of the invention (either in a liquid, lyophilized or final dosage form (e.g. capsule) is suitable for storage at about 2° C. to about 4° C., 15° C. or at ambient temperature. In some embodiments, the formulations are stored with a desiccant molecular sieve pack to reduce moisture during storage. In some embodiments, the formulation is stored in a container, e.g., a bottle or other suitable container, with a desiccant molecular sieve pack to reduce moisture during storage.

The formulations of the present invention (either in a liquid, lyophilized or final dosage form (e.g. capsule) provide for the chemical stability of the formulated antibody and other optional active agents of the formulation. “Stability” and “stable” in this context refers to the resistance of antibody and other optional active agents to chemical degradation and physical changes such as settling, precipitation, aggregation under given manufacturing, and preparation, transportation and storage conditions. The “stable” formulations of the invention also preferably retain at least 90%, 95%, 98%, 99%, or 99.5% of a starting or reference amount under given manufacturing, preparation, transportation, and/or storage conditions. The amount of antibody and other optional active agents can be determined using any art-recognized method, for example, as UV-Vis spectrophotometry and high pressure liquid chromatography (HPLC), or SDS-PAGE.

The antibody formulations of the invention (either in a liquid, lyophilized or final dosage form (e.g. capsule) are stable for at least 3 months at either 4° C., 15° C., or ambient temperature. The formulations are stable for more than 3 months at either 4° C. or 15° C., for example, at least 4 months, at least 5 months, at least 6 months, at least 7 months, at least 8 months, at least 9 months, at least 10 months, at least 11 months, at least 12 months, at least 18 months, at least 24 months and/or greater than 24 months at either 4° C., 15° C., or ambient temperature.

The antibody formulations of the invention (either in a liquid, lyophilized or final dosage form (e.g. capsule) have a purity of at least 90%, 91%, 92% 95%, 95%, 97%, 985, 99% or more IgG as heavy and light chains.

The antibody formulations of the invention (either in a liquid, lyophilized or final dosage form (e.g. capsule) have less than 5%, 4%, 3%, 2%, 1% total impurities.

The antibody formulations of the invention (either in a liquid, lyophilized or final dosage form (e.g. capsule) have least 90%, 91%, 92% 95%, 95%, 97%, 985, 99% or more IgG monomers.

The antibody formulations of the invention (either in a liquid, lyophilized or final dosage form (e.g. capsule) have less than 5%, 4%, 3%, 2%, 1%, 0.9%, 0.8%, 0.7%, 0.6%, 0.5%, 0.4%, 0.3%, 0.2%, 0.1% total IgG aggregates.

Dosage Forms

The formulations of the invention are specifically formulated for enteral or parenteral, administration.

For enteral administration, i.e., oral, the formulations may be a capsule or a tablet.

The capsules include soft gel capsules or hard shell capsules. Soft gel capsules are a soft gel or gelatin or gelatin-like material. The hard shell or soft gel capsules are HPMC capsules. The capsules, soft gel or hard shell may be filed with a liquid antibody formulation or a powdered, e.g, lyophilized, anti-CD3 formulation. Exemplary, liquid and powdered anti-CD3 formulations are described above.

In some embodiments, each capsule includes a sufficient enteric coating to bypass stomach acidity. Any suitable enteric coating can be used in the oral anti-CD3 antibody formulations, including, by way of non-limiting example, enteric coatings such as Eudragit®, e.g., Eudragit® L 30 D/L 100-55, which releases the anti-CD3 antibody at a pH above 4 or 5.

In some embodiments, each capsule in the oral anti-CD3 antibody formulation comprises a soft gel or gelatin or gelatin-like material having a size in the range of 0 to 2, e.g., a size 0, a size 1, and/or a size 2.

In some embodiments capsule in the oral anti-CD3 antibody formulation is a liquid-filled hard capsule (LFHC). Any suitable LFHC can be used in the oral anti-CD3 antibody formulation of the disclosure, including, by way of non-limiting example, Licaps® and other LFHC by Capsugel®.

In some embodiments, each liquid-filled capsule in the oral anti-CD3 antibody formulation contains a volume less than about 1000 μL, e.g., less than about 75 μL, and/or less than about 500 μL. In some embodiments, each liquid-filled capsule in the oral anti-CD3 antibody formulation contains a volume in a range from about 50 μL to about 1000 μL, from about 100 μL to about 1000 μL, from about 200 μL to about 1000 μL, from about 250 μL to about 1000 μL, from about 50 μL to about 500 μL, from about 100 μL to about 500 μL, from about 200 μL to about 500 μL, and/or from about 250 μL to about 500 μL.

A preferred oral formulation includes an enteric coated oral capsule containing an anti-CD3 antibody lyophilized formulation having a unit dose of about 0.1 mg to 10 mg of an anti-CD3 antibody or antigen binding fragment thereof and about 0.58 mg of sodium acetate trihydrate, about 1.25 mg sodium chloride, about 0.034 mg polysorbate 80, about 34 mg trehalose and about 0.17 mg methionine per 1 mg of anti-CD3 antibody or antigen binding fragment thereof. Optionally, the enteric-coated oral capsule further includes 0.17 mg EDTA per 1 mg of anti-CD3 antibody or antigen binding fragment thereof. The unit dosed is 0.5 mg, 2.5 mg or 5.0 mg.

Another preferred oral formulation includes an enteric coated oral capsule containing an anti-CD3 antibody liquid formulation comprising a unit dose of about 0.1 mg to 10 mg of an anti-CD3 antibody or antigen binding fragment thereof, 25 mM sodium acetate trihydrate, 125 mM sodium chloride, 0.02% polysorbate 80 (w/v), 20% trehalose (w/v), and 0.1% methionine (w/v). Optionally, the enteric-coated oral capsule further includes 0.1% EDTA The unit dosed is 0.5 mg, 2.5 mg or 5.0 mg.

In some embodiments, the anti-CD3 antibody formulation is a subcutaneous formulation. In some embodiments, the subcutaneous anti-CD3 antibody formulation is housed in a sealed vial or other container.

In some embodiments, the subcutaneous anti-CD3 antibody formulation includes an anti-CD3 antibody, at least one salt, at least one surfactant, and a volume of water necessary to bring the formulation to the desired injection volume.

In some embodiments, the subcutaneous anti-CD3 antibody formulation includes about 2 mg/mL of the anti-CD3 antibody, about 7.31 mg sodium chloride, about 3.40 mg sodium acetate trihydrate, about 0.20 mg Polysorbate 80, and water in an amount to bring the formulation volume up to 1 ml for the desired injection volume. The subcutaneous antibody formulation should be at a pH in the range of about 4 to 6.

In some embodiments, the subcutaneous anti-CD3 antibody formulation is stored in a vial or other suitable container under refrigeration, e.g., in the range of about 2° C. to about 8° C. In some embodiments, the subcutaneous anti-CD3 antibody formulation is not shaken. In some embodiments, the subcutaneous anti-CD3 antibody formulation is not frozen. In some embodiments, the subcutaneous anti-CD3 antibody formulation is diluted prior to administration.

In some embodiments, the subcutaneous anti-CD3 antibody formulation is administered at a dose in a range from about 1 mg/60 kg body weight to about 10 mg/60 kg body weight.

It will be appreciated that administration of therapeutic entities in accordance with the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman WN “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Therapeutic Administration

Therapeutic formulations provided herein, which include an anti-CD3 antibody, alone or together with a compound that improves gut barrier function and/or an anti-inflammatory compound are used to treat or alleviate a symptom associated with a gastrointestinal disorder disorder, such as, for example, an autoimmune disease or an inflammatory disorder of the gastrointestinal system. S

Specifically, the formulation disclosed are also used to treat or alleviate a symptom associated CeD and CD.

The therapeutic formulations are administered to a subject suffering from an gastrointestinal disorder, such as an autoimmune disease or an inflammatory disorder. A subject suffering from an autoimmune disease, an inflammatory disorder, neurodegenerative disorder or cancer is identified by methods known in the art.

Administration of the therapeutic formulations to a patient suffering from an gastrointestinal disorder such as an autoimmune disease or an inflammatory disorder, is considered successful if any of a variety of laboratory or clinical results is achieved. For example, treatment considered successful if one or more of the symptoms associated with the disorder is alleviated, reduced, inhibited or does not progress to a further, i.e., worse, state.

In another embodiment, the therapeutic formulations provided herein are used in the treatment, diagnosis and/or prevention of Crohn's disease. Crohn's disease is the chronic inflammation and irritation of the intestines. Crohn's disease is associated with symptoms such as abdominal pain, diarrhea, weight loss, poor appetite, fever, night sweats, rectal pain, and rectal bleeding. The therapeutic formulations provided herein are administered to a subject that is suffering from, has been diagnosed with, or is predisposed to Crohn's disease. The antibody formulations provided herein are administered at a dosage that is sufficient to alleviate at least one symptom of Crohn's disease, to treat Crohn's disease, to prevent Crohn's disease, and/or to prevent Crohn's disease from progressing to a further disease state in a subject.

The therapeutic formulations is used to activate regulatory T-cells (Tregs).

In another embodiment used herein, therapeutic formulations composition is administered to human subjects to prevent, reduce or decrease the recruitment of immune cells into human tissues. A therapeutic formulation used herein is administered to a subject in need thereof to prevent and treat conditions associated with abnormal or deregulated immune cell recruitment into tissue sites of human disease.

In another embodiment used herein, a therapeutic formulation composition is administered to human subjects to prevent, reduce or decrease the extravasation and diapedesis of immune cells into human tissues. Thus, the therapeutic formulations used herein are administered to prevent and/or treat conditions associated with abnormal or deregulated immune cell infiltration into tissue sites of human disease.

In another embodiment used herein, the therapeutic formulations composition is administered to human subjects to prevent, reduce or decrease the effects mediated by the release of cytokines within the human body. The term “cytokine” refers to all human cytokines known within the art that bind extracellular receptors upon the cell surface and thereby modulate cell function, including but not limited to IL-2, IFN-g, TNF-α, IL-4, IL-5, IL-6, IL-9, IL-10, and IL-13.

In another embodiment used herein, the therapeutic formulation is administered to human subjects to prevent, reduce or decrease the effects mediated by the release of cytokine receptors within the human body. The term “cytokine receptor” refers to all human cytokine receptors within the art that bind one or more cytokine(s), as defined herein, including but not limited to receptors of the aforementioned cytokines. Thus, the therapeutic formulation used herein is administered to treat and/or prevent conditions mediated through abnormal activation, binding or ligation of one or more cytokine receptor(s) within the human body. It is further envisioned that administration of the therapeutic formulation in vivo will deplete the intracellular signaling mediated by cytokine receptor(s) within such human subject.

In one aspect used herein, the therapeutic formulation composition is administered to a human individual upon decrease of pancreatic beta-cell function therein. In one embodiment, the individual is tested for beta-cell function, insulin secretion or c-peptide levels as are known within the art. Subsequently, upon notice of sufficient decrease of either the indicator, the human individual is administered with a sufficient dosage regimen of the therapeutic formulation to prevent further progression of autoimmune destruction of beta-cell function therein.

Preferably, the therapeutic antibody formulations provided herein are administered to a subject oral, subcutaneously or nasally. Other routes of administration are contemplated. For example, the antibody formulations are administered intravenously, intramuscularly, or any combination of these routes of administration.

Combination Therapy

The anti-CD3 antibody formulation is administered during and/or after treatment in combination with one or more additional agents such as, for example, an compound that that improves gut barrier function and/or reduces gut permeability and/or an anti-inflammatory compound.

In some embodiments, the anti-CD3 antibody and the additional agent (3) are formulated into a single therapeutic composition, and the anti-CD3 antibody and additional agent are administered simultaneously.

Alternatively, the anti-CD3 antibody and additional agent(s) are separate from each other, e.g., each is formulated into a separate therapeutic composition, and the anti-CD3 antibody and the additional agent(s) are administered simultaneously, or the anti-CD3 antibody and the additional agent(s) are administered at different times during a treatment regimen. For example, the anti-CD3 antibody is administered prior to the administration of the additional agent(s), the anti-CD3 antibody is administered subsequent to the administration of the additional agent(s), or the anti-CD3 antibody and the additional agent(s) are administered in an alternating fashion. As described herein, the anti-CD3 antibody and additional agent(s) are administered in single doses or in multiple doses.

In some embodiments, the anti-CD3 antibody and the additional agent(s) are administered simultaneously. For example, the anti-CD3 antibody and the additional agent(s) can be formulated in a single composition or administered as two or more separate compositions. In some embodiments, the anti-CD3 antibody and the additional agent(s) are administered sequentially, or the anti-CD3 antibody and the additional agent are administered at different times during a treatment regimen.

Administration of an anti-CD3 antibody alone or in combination with one or more additional agents, to a patient suffering from an inflammatory diseases (e.g., inflammatory disorders of the GI tract), gastrointestinal disorders (e.g., CeD, CD, ulcerative colitis, irritable bowel syndrome, inflammatory bowel disease, or colon cancer), autoimmune disease (e.g. diabetes), cancer (e.g., GI cancer), NASH, bile disorders, or liver diseases is considered successful if any of a variety of laboratory or clinical objectives is achieved.

For example, administration of an anti-CD3 antibody, alone or in combination with one or more additional agents, to a patient suffering from an inflammatory diseases (e.g., inflammatory disorders of the GI tract), GI disorders (e.g., celiac disease, Crohn's disease, ulcerative colitis, irritable bowel syndrome, inflammatory bowel disease, or colon cancer), autoimmune disease (e.g. diabetes), cancer (e.g., gastrointestinal cancer), NASH, bile disorders, or liver diseases is considered successful if one or more of the symptoms associated with the disease or disorder is alleviated, reduced, inhibited or does not progress to a further, i.e., worse, state.

Administration of an anti-CD3 antibody, alone or in combination with one or more additional agents, to a patient suffering from an inflammatory diseases (e.g., inflammatory disorders of the gastrointestinal tract), gastrointestinal disorders (e.g., celiac disease, Crohn's disease, ulcerative colitis, irritable bowel syndrome, inflammatory bowel disease, or colon cancer), autoimmune disease (e.g. diabetes), cancer (e.g., gastrointestinal cancer), NASH, bile disorders, or liver diseases is considered successful if the disease or disorder enters remission or does not progress to a further, i.e., worse, state.

Additional agents suitable for use with the compositions and methods of the present invention include for example retinoic acid such as all-trans-retinoic acid, 9-cis-retinoic acid, or 13-cis-retinoic acid, a GC-C receptor agonist such as guanylin, uroguanylin, lymphoguanylin, heat-stable enterotoxin of E. coli (ST), linaclotide, plecanatide or dolcanatide, heparin, lubiprostone, glutamine, GLP-2 peptide, a zolulin peptide inhibitor, a zonulin antagonist, larazotide, a probiotic bacteria or butyric acid. Preferably, the GC-C receptor agonist is Linzess® or Trulance®.

Additional agents suitable for use with the compositions and methods of the present invention includes anti-inflammatory compounds. Anti-inflammatory compounds include for example a monoclonal antibody such as a monoclonal antibody specific for IL-17, TNF-a, α4-integrin, α4-β7 integrin, IL-12, or IL-23, Remicade®, a JNK inhibitor, a PI3K pathway inhibitor, an AKT inhibitor, a mTOR inhibitor, mesalamine, uceris or a sphinosine-1-phosphate kinase inhibitor. Preferred, monoclonal antibodies include Humira®, Tysabri®, Entyvio®, or Stelara®.

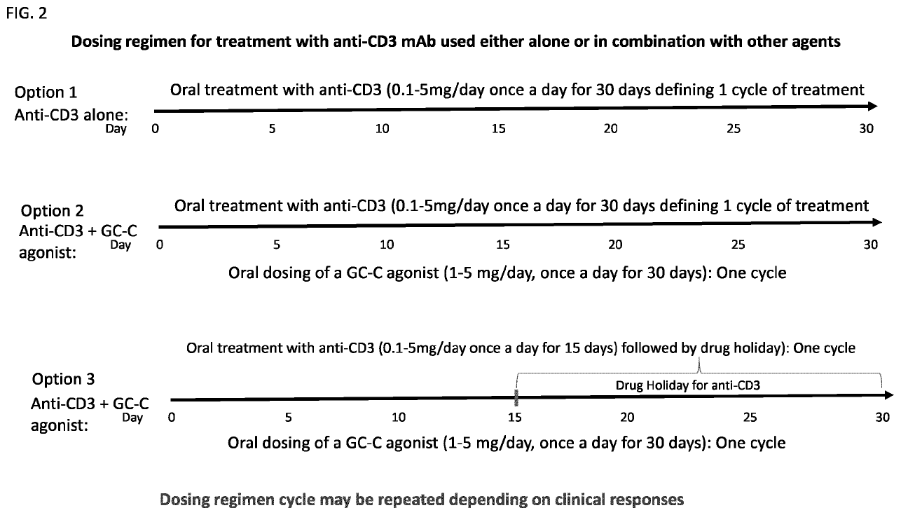

In some embodiments, the combination therapy that includes an anti-CD3 antibody and at least a second therapeutic agent is administered in a dosing regimen shown in FIG. 2. In some embodiments, the combination therapy that includes an anti-CD3 antibody and at least a second therapeutic agent is administered in a dosing regimen shown in FIG. 2, and the dosing regimen is repeated. In some embodiments, the combination therapy that includes an anti-CD3 antibody and at least a second therapeutic agent is administered in a dosing regimen shown in FIG. 2, and the dosing regimen is repeated after the drug holiday period. In some embodiments, the combination therapy that includes an anti-CD3 antibody and at least a second therapeutic agent is administered in a dosing regimen shown in FIG. 2, and the drug holiday cycle is repeated. In some embodiments, the combination therapy that includes an anti-CD3 antibody and at least a second therapeutic agent is administered in a dosing regimen shown in FIG. 2, and the dosing regimen and drug holiday cycle are repeated.

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the term “antibody” refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, Fab, Fab, and F(ab′)2 fragments, and an Fab expression library. By “specifically bind” or “immunoreacts with” is meant that the antibody reacts with one or more antigenic determinants of the desired antigen and does not react (i.e., bind) with other polypeptides or binds at much lower affinity (K_{d}>10⁻⁶) with other polypeptides.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ea., 2nd ed. Raven Press, N.Y. (1989)). The variable regions of each light/heavy chain pair form the antibody binding site.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

As used herein, the term “epitope” includes any protein determinant capable of specific binding to an immunoglobulin, a scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≤1 μM; preferably ≤100 nM and most preferably ≤10 nM.

As used herein, the terms “immunological binding” and “immunological binding properties” and “specific binding” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides are quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody of the present invention is said to specifically bind to a CD3 epitope when the equilibrium binding constant (K_{d}) is ≤1 μM, preferably ≤100 nM, more preferably ≤10 nM, and most preferably ≤100 pM to about 1 pM, as measured by assays such as radioligand binding assays or similar assays known to those skilled in the art.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family.

The term “agent” is used herein to denote a chemical compound, a mixture of chemical compounds, a biological macromolecule, or an extract made from biological materials.

The term patient includes human and veterinary subjects.

The disclosure also includes Fv, Fab, Fab, and F(ab′)₂ anti-CD3 antibody fragments, single chain anti-CD3 antibodies, bispecific anti-CD3 antibodies, heteroconjugate anti-CD3 antibodies, trispecific antibodies, immunoconjugates and fragments thereof.

Bispecific antibodies are antibodies that have binding specificities for at least two different antigens. In the present case, one of the binding specificities is for CD3. The second binding target is any other antigen, and advantageously is a cell-surface protein or receptor or receptor subunit.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The disclosure having now been described by way of written description, those of skill in the art will recognize that the disclosure can be practiced in a variety of embodiments and that the foregoing description and examples below are for purposes of illustration and not limitation of the claims that follow.

### OTHER EMBODIMENTS

While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A method for treating a disorder comprising administering an oral dosage form of an anti-CD3 antibody and a compound that improves gut barrier function and/or an anti-inflammatory compound.

2. The method of claim 1, wherein the disorder is an inflammatory disease, a gastrointestinal disorder, an autoimmune disease or cancer.

3. The method of claim 2, wherein the inflammatory disorder is an inflammatory disorder of the gastrointestinal tract.

4. The method of claim 2, wherein the gastrointestinal disorder is a gastrointestinal cancer.

5. The method claim 2, wherein the gastrointestinal disorder is celiac disease, Crohn's disease, ulcerative colitis, irritable bowel syndrome, inflammatory bowel disease, or colon cancer.

6. The method of claim 2, wherein the autoimmune disease is diabetes.

7. The method of claim 1, wherein the disorder is NASH, a bile acid disorder or liver disease.

8. The method of any one of the preceding claims, wherein the compound that improves gut barrier function reduces gut permeability.

9. The method of any one of the preceding claims, wherein the compound that improves gut barrier function is a retinoic acid, a GC-C receptor agonist, heparin, lubiprostone, glutamine, GLP-2 peptide, a zolulin peptide inhibitor, a zonulin antagonist, larazotide, a probiotic bacteria or butyric acid.

10. The method of claim 9, wherein the retinoic acid is all-trans-retinoic acid, 9-cis-retinoic acid, or 13-cis-retinoic acid.

11. The method of claim 9, wherein GC-C receptor agonist is guanylin, uroguanylin, lymphoguanylin, heat-stable enterotoxin of E. coli (ST), linaclotide, plecanatide or dolcanatide.

12. The method of claim 11, wherein the linaclotide is Linzess®.

13. The method of claim 11, wherein plecanatide is Trulance®.

14. The method of claim 1, wherein the anti-inflammatory compound is a monoclonal antibody specific for IL-17, TNF-a, α4-integrin, α4-β7 integrin, IL-12, or IL-23.

15. The method of claim 1, wherein the an anti-inflammatory compound is Remicade®, a JNK inhibitor, a PI3K pathway inhibitor, an AKT inhibitor, a mTOR inhibitor, mesalamine, Uceris or a sphinosine-1-phosphate kinase inhibitor.

16. The method of any one of claim 9, wherein the retinoic acid is administered at a daily dose of about 15-45 mg/m².

17. The method of claim 14, wherein the retinoic acid is administered at a daily dose of about 45 mg/m².

18. The method of claim 15, wherein the daily dose is administered in two equally divided doses.

19. The method of claim 14, wherein the retinoic acid is administered at a dose of about 22.5 mg/m² twice daily.

20. The method of any one of the preceding claims, wherein the GC-C receptor agonist is administered at a daily dose of about 1 to about 10 mg.

21. The method of any one of the preceding claims, wherein the GC-C receptor agonist is administered at a daily dose of 3 mg or 6 mg.

22. The method of any one of the preceding claims, wherein the GC-C agonist is administered once daily.

23. The method of anyone of the preceding claims, wherein the anti-CD3 antibody is fully human, humanized or murine.

24. The method of any one of the preceding claims, wherein the anti-CD3 antibody administered at a daily dose of about 0.1 mg to about 10 mg.

25. The method of any one of the preceding claims, wherein the anti-CD3 antibody is administered for a treatment cycle of 30 days.

26. The method of any one of the preceding claims, wherein the anti-CD3 antibody is administered for a treatment cycle comprising an on period of 15 days and an off period of 15 days.

27. The method of any one of the preceding claims, wherein the anti-CD3 antibody and the compound that increases gut barrier function are administered for a treatment cycle of 30 days.

28. The method of any one of the preceding claims, wherein the anti-CD3 antibody and the compound that increases gut barrier function are administered for a treatment cycle, the treatment cycle comprising an on period of 15 days and an off period of 15 days for the anti-CD3 antibody and an on period of 30 days for the compound that increases gut barrier function.

29. The method of any one of the preceding claims, wherein the anti-CD3 antibody, the compound that increases gut barrier function and the GC-C receptor agonist, are administered for a treatment cycle or 30 days.

30. The method of any one of the preceding claims, wherein the anti-CD3 antibody, the compound that increases gut barrier function and the GC-C receptor agonist, are administered for a treatment cycle, the treatment cycle comprising an on period of 15 days and an off period of 15 days for the anti-CD3 antibody or antigen-binding fragment thereof, an on period of 30 days for the GC-C agonist, and an on period of 30 days for the retinoic acid.

31. The method of any one of claims 25-30, wherein the each treatment cycle is repeated 2, 3, 4, 5, 6, 7, 8, 9, or 10 times.

32. The method of any one of the preceding claims, wherein the oral dosage form of the anti-CD3 antibody is an enteric polymer-coated oral dosage form.

33. The method of claim 32, wherein the enteric polymer-coated oral dosage form is a liquid-filled capsule.

34. The method of claim 33, wherein the enteric polymer-coated, liquid-filled oral capsule comprises a liquid formulation comprising a unit dose of about 0.1 mg to 10 mg of an anti-CD3 antibody about 10 mM to about 500 mM sodium acetate trihydrate, about 10 mM to 500 mM sodium chloride, and about 0.01% to 1% polysorbate 80 (w/v)

35. The method of claim 34, wherein the enteric polymer-coated oral dosage form is a powder-filled capsule.

36. The method of claim 35, wherein the enteric polymer-coated, powder-filled oral capsule comprises a lyophilized powder formulation comprising a unit dose of about 0.1 mg to 10 mg of an anti-CD3 antibody and one or more pharmaceutically acceptable excipients.

37. The method of any of the proceeding claims, wherein the anti-CD3 antibody comprises:
(i) a heavy chain comprising a variable heavy chain complementarity determining region 1 (VH CDR1) comprising the amino acid sequence of GYGMH (SEQ ID NO: 1), a variable heavy chain complementarity determining region 2 (VH CDR2) comprising the amino acid sequence of VIWYDGSKKYYVDSVKG (SEQ ID NO: 2), a variable heavy chain complementarity determining region 3 (VH CDR3) comprising the amino acid sequence of QMGYWHFDL (SEQ ID NO: 3); and a light chain comprising a variable light chain complementarity determining region 1 (VL CDR1) comprising the amino acid sequence of RASQSVSSYLA (SEQ ID NO: 4), a variable light chain complementarity determining region 2 (VL CDR2) comprising the amino acid sequence of DASNRAT (SEQ ID NO: 5), a variable light chain complementarity determining region 3 (VL CDR3) comprising the amino acid sequence of QQRSNWPPLT (SEQ ID NO: 6);
(ii) a heavy chain comprising a VH CDR1 comprising the amino acid sequence SYGMH (SEQ ID NO: 7); a VH CDR2 comprising the amino acid sequence of IIWYDGSKKNYADSVKG (SEQ ID NO: 8); a VH CDR3 comprising the amino acid sequence of GTGYNWFDP (SEQ ID NO: 9); and a light chain comprising a VL CDR1 comprising the amino acid sequence of RASQSVSSSYLA (SEQ ID NO: 10), RASQGISSALA (SEQ ID NO: 11) or WASQGISSYLA (SEQ ID NO: 12); a VL CDR2 comprising the amino acid sequence of GASSRAT (SEQ ID NO: 13), YASSLQS (SEQ ID NO: 14), or DASSLGS (SEQ ID NO: 15); and a VL CDR3 comprising the amino acid sequence of QQYGSSPIT (SEQ ID NO: 16) or QQYYSTLT (SEQ ID NO: 17); and
(iii) a heavy chain comprising a VH CDR1 comprising the amino acid sequence SYGMH (SEQ ID NO: 7); a VH CDR2 comprising the amino acid sequence of AIWYNGRKQDYADSVKG (SEQ ID NO: 18); a VH CDR3 comprising the amino acid sequence of GTGYNWFDP (SEQ ID NO: 9); and a light chain comprising a VL CDR1 comprising the amino acid sequence of RASQSVSSYLA (SEQ ID NO: 4) or RASQGISSALA (SEQ ID NO: 11); a VL CDR2 comprising the amino acid sequence of DASNRAT (SEQ ID NO: 5) or DASSLES (SEQ ID NO: 19); and a VL CDR3 comprising the amino acid sequence of QQRSNWPWT (SEQ ID NO: 20) or QQFNSYPIT (SEQ ID NO: 21).

38. The method of any of the proceeding claims, wherein the anti-CD3 antibody o comprises a heavy chain variable region (VH) having the amino acid sequence of SEQ ID NOs: 22 (28F11 VH), 24 (23F10 VH), 26 (27H5 VH), or 32 (15C3 VH), and a light chain variable region (VL) having the amino acid sequence of SEQ ID NOs: 23 (28F11 VL), 25 (23F10 VL), 27 (27H5 VL1), 28 (27H5 VL2), 29 (27H5 VL3), 30 (27H5 VL4), 31 (27H5 VL5), 33 (15C3 VL1), or 34 (15C3 VL2).

39. The method of any one of claims 1-36, wherein the anti-CD3 antibody comprises a heavy chain variable region (VH) having the amino acid sequence of SEQ ID NO: 22 (28F11 VH) and a light chain variable region (VL) having the amino acid sequence of SEQ ID NO: 23 (28F11 VL).

40. The method of any one of claims 1-36, wherein the anti-CD3 antibody thereof comprises a heavy chain variable region (VH) having the amino acid sequence of SEQ ID NO: 24 (23F10 VH) and a light chain variable region (VL) having the amino acid sequence of SEQ ID NO: 25 (23F10 VL).

41. The method of any one of claims 1-36, wherein the anti-CD3 antibody comprises a heavy chain variable region (VH) having the amino acid sequence of SEQ ID NO: 26 (27H5 VH) and a light chain variable region (VL) having the amino acid sequence of SEQ ID NOs: 27 (27H5 VL1), 28 (27H5 VL2), 29 (27H5 VL3), 30 (27H5 VL4), 31 (27H5 VL5).

42. The method of any one of claims 1-36, wherein the anti-CD3 thereof comprises a heavy chain variable region (VH) having the amino acid sequence of SEQ ID NO: 32 (15C3 VH), and a light chain variable region (VL) having the amino acid sequence of SEQ ID NOs: 33 (15C3 VL1) or 34 (15C3 VL2).

43. The method of any one of the preceding claims, wherein the anti-CD3 antibody or antigen-binding fragment thereof comprises a framework 2 region (FWR2) comprising the amino acid sequence WVRQAPGKGLEWV (SEQ ID NO: 35).

44. The method of any one of the preceding claims, wherein the anti-CD3 antibody or antigen-binding fragment thereof comprises a framework 3 region (FRW3) comprising the amino acid sequence RFTISRDNSKNTLYLQMNSLRAEDTAVYYCA (SEQ ID NO:36).

45. The method of any one of the preceding claims, wherein the anti-CD3 antibody is an IgG1 isotype.

46. The method of any one of the preceding claims, wherein the anti-CD3 antibody or antigen-binding fragment thereof includes a mutation in the heavy chain at an amino acid residue at position 234, 235, 265, or 297 or combinations thereof, and reduces the release of cytokines from a T cell, wherein the amino acid residue positions are numbered according to Kabat

47. The method of claim 46, wherein said mutation results in an alanine or glutamic acid residue at said position.

48. The method of any one of claims 1-47, wherein the anti-CD3 antibody or antigen-binding fragment thereof is an IgG1 isotype and contains at least a first mutation at position 234 and a second mutation at position 235, wherein said first mutation results in an alanine residue at position 234 and said second mutation results in a glutamic acid residue at position 235, wherein the amino acid residue positions are numbered according to Kabat.


SEQUENCE LISTING (36 sequences)

SEQ ID NO: 1 - protein, 5 aa, Homo sapiens
GYGMH

SEQ ID NO: 2 - protein, 17 aa, Homo sapiens
VIWYDGSKKYYVDSVKG

SEQ ID NO: 3 - protein, 9 aa, Homo sapiens
QMGYWHFDL

SEQ ID NO: 4 - protein, 11 aa, Homo sapiens
RASQSVSSYLA

SEQ ID NO: 5 - protein, 7 aa, Homo sapiens
DASNRAT

SEQ ID NO: 6 - protein, 10 aa, Homo sapiens
QQRSNWPPLT

SEQ ID NO: 7 - protein, 5 aa, Homo sapiens
SYGMH

SEQ ID NO: 8 - protein, 17 aa, Homo sapiens
IIWYDGSKKNYADSVKG

SEQ ID NO: 9 - protein, 9 aa, Homo sapiens
GTGYNWFDP

SEQ ID NO: 10 - protein, 12 aa, Homo sapiens
RASQSVSSSYLA

SEQ ID NO: 11 - protein, 11 aa, Homo sapiens
RASQGISSALA

SEQ ID NO: 12 - protein, 11 aa, Homo sapiens
WASQGISSYLA

SEQ ID NO: 13 - protein, 7 aa, Homo sapiens
GASSRAT

SEQ ID NO: 14 - protein, 7 aa, Homo sapiens
YASSLQS

SEQ ID NO: 15 - protein, 7 aa, Homo sapiens
DASSLGS

SEQ ID NO: 16 - protein, 9 aa, Homo sapiens
QQYGSSPIT

SEQ ID NO: 17 - protein, 8 aa, Homo sapiens
QQYYSTLT

SEQ ID NO: 18 - protein, 17 aa, Homo sapiens
AIWYNGRKQDYADSVKG

SEQ ID NO: 19 - protein, 7 aa, Homo sapiens
DASSLES

SEQ ID NO: 20 - protein, 9 aa, Homo sapiens
QQRSNWPWT

SEQ ID NO: 21 - protein, 9 aa, Homo sapiens
QQFNSYPIT

SEQ ID NO: 22 - protein, 118 aa, Homo sapiens [sequence omitted]

SEQ ID NO: 23 - protein, 108 aa, Homo sapiens [sequence omitted]

SEQ ID NO: 24 - protein, 118 aa, Homo sapiens
QVQLVQSGGGVVQSGRSLRLSCAASGFKFSGYGMHWVRQAPGKGLEWVAVIWYDGSKKYYVDSVKGRFTISRDNSKNTLYLQMNSLRGEDTAVYYCARQMGYWHFDLWGRGTLVTVSS

SEQ ID NO: 25 - protein, 108 aa, Homo sapiens [sequence omitted]

SEQ ID NO: 26 - protein, 118 aa, Homo sapiens
QVQLVESGGGVVQPGRSLRLSCAASGFTFRSYGMHWVRQAPGKGLEWVAIIWYDGSKKNYADSVKGRFTISRDNSKNTLYLQMNSLRAEDTAVYYCARGTGYNWFDPWGQGTLVTVSS

SEQ ID NO: 27 - protein, 108 aa, Homo sapiens
EIVLTQSPRTLSLSPGERATLSCRASQSVSSSYLAWYQQKPGQAPRLLIYGASSRATGIPDRFSGSGSGTDFTLTISRLDPEDFAVYYCQQYGSSPITFGQGTRLEIK

SEQ ID NO: 28 - protein, 106 aa, Homo sapiens
DILMTQSPSSLSASVGDRVTITCRASQGISSALAWYQQKPGKAPKLLIYYASSLQSGVPSRFSGSGSGTDYTLTISSLQPEDFATYYCQQYYSTLTFGGGTKVEIK

SEQ ID NO: 29 - protein, 106 aa, Homo sapiens
DIVMTQSPSSLSASVGDRVTITCRASQGISSALAWYQQKPGKAPKLLIYDASSLGSGVPSRFSGSGSGTDFTLTISSLQPEDFATYYCQQYYSTLTFGGGTKVEIK

SEQ ID NO: 30 - protein, 106 aa, Homo sapiens
DIQMTQSPFSLSASVGDRVTITCWASQGISSYLAWYQQKPAKAPKLFIYYASSLQSGVPSRFSGSGSGTDYTLTISSLQPEDFATYYCQQYYSTLTFGGGTKVEIK

SEQ ID NO: 31 - protein, 106 aa, Homo sapiens
DIEMTQSPFSLSASVGDRVTITCWASQGISSYLAWYQQKPAKAPKLFIYYASSLQSGVPSRFSGSGSGTDYTLTISSLQPEDFATYYCQQYYSTLTFGGGTKVEIK

SEQ ID NO: 32 - protein, 118 aa, Homo sapiens
QVQLVQSGGGVVQPGRSLRLSCVASGFTFSSYGMHWVRQAPGKGLEWVAAIWYNGRKQDYADSVKGRFTISRDNSKNTLYLQMNSLRAEDTAVYYCTRGTGYNWFDPWGQGTLVTVSS

SEQ ID NO: 33 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 34 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIK

SEQ ID NO: 35 - protein, 13 aa, Homo sapiens
WVRQAPGKGLEWV

SEQ ID NO: 36 - protein, 31 aa, Homo sapiens
RFTISRDNSKNTLYLQMNSLRAEDTAVYYCA